(19) United States
(12) Patent Application Publication
(10) Pub. No.: US 2016/0015711 A1
(43) Pub. Date: Jan. 21, 2016

(54) CALCIUM PTERIN-6-CARBOXYLATE (CaPTERIN-6-COOH) AS A NOVEL IMMUNO-THERAPEUTIC

(71) Applicant: SanRx Pharmaceuticals, Inc., La Jolla, CA (US)

(72) Inventor: Phillip B.B. MOHENO, San Diego, CA (US)

(21) Appl. No.: 14/749,532

(22) Filed: Jun. 24, 2015

Related U.S. Application Data
(60) Provisional application No. 62/017,139, filed on Jun. 25, 2014.

(51) Int. Cl. A61K 31/519; A61K 31/167

(52) U.S. Cl. CPC A61K 31/519; A61K 31/167

(57) ABSTRACT

Provided herein are methods of treating an inflammatory-based disease or disorder in a subject by administering a composition comprising CaPterin-6-COOH.

### CROSS-REFERENCE

This application claims the benefit of priority from U.S. Provisional Application Ser. No. 62/017,139, filed Jun. 25, 2014, which is incorporated herein by reference in its entirety.

### BACKGROUND OF THE INVENTION

Previously, it has been reported that Pterins are an immuno-modulator present in the blood and tissues of mammals [5-11]. Pterin is excreted in the urine of cancer patients in elevated amounts relative to normal persons [12], implicating elevated amounts internally. When combined with calcium, oral Pterin demonstrates anti-tumorgenic [1-3], anti-viral such as hepatitis B [13], anti-diabetic [14], and anti-mycobacterial (BCG) activity in an in vitro model of tuberculosis [15]. In several of these studies it was reported that a promising new cancer therapeutic, dipterinyl calcium pentahydrate (DCP), a dimer of pterin linked together with calcium (FIG. 1), shows the same immuno-modulatory, anti-tumor, anti-infective, and anti-diabetic activities as the monomer CaPterin. Under pH <4 conditions, DCP breaks down to form CaPterin.

### SUMMARY OF THE INVENTION

Calcium Pterin-6-carboxylate has been identified as the most likely primary active moiety generated by Calcium Folate in solution and furthermore, CaPterin-6-COOH has been observed to exhibit a similar and more potent biological efficacy profile as compared to CaPterin, DCP, and CaFolate in a canine arthritic case study.

In one embodiment is a method of treating an inflammatory-based disease or disorder in a subject by administering a composition comprising CaPterin-6-COOH. In another embodiment CaPterin-6-COOH has a greater therapeutic efficacy as compared to calcium pterin. In another embodiment CaPterin-6-COOH has a greater therapeutic efficacy as compared to dipterinyl calcium pentahydrate. In another embodiment CaPterin-6-COOH has a greater therapeutic efficacy as compared to calcium folate.

In another embodiment is a method of administering a composition comprising CaPterin-6-COOH through oral, parenteral, intravenous, subcutaneous, intrathecal, intramuscular, buccal, intranasal, epidural sublingual, pulmonary, local, rectal, or transdermal administration.

In another embodiment is a method of administering a composition comprising CaPterin-6-COOH to treat arthritis. In another embodiment the arthritis is osteoarthritis, rheumatoid arthritis, gout, psoriatic arthritis, lupus, or septic arthritis.

In another embodiment is a method of producing CaPterin-6-COOH from folic acid and a calcium source. In another embodiment the calcium source is calcium chloride. In another embodiment a method of administering CaPterin-6-COOH comprises administering to a subject folic acid and a calcium source, wherein CaPterin-6-COOH is produced in the subject.

In another embodiment CaPterin-6-COOH modulates indoleamine 2,3-dioxygenase pathways. In another embodiment a composition comprising CaPterin-6-COOH inhibits indoleamine 2,3-dioxygenase.

In another embodiment CaPterin-6-COOH is produced by folic acid exposure to UV light yielding intermediates p-aminobenzoylglutamate and 6-FP, which in turn is oxidized to CaPterin-6-COOH.

In another embodiment is a method of administering CaPterin-6-COOH as a single active agent to a subject. In another embodiment the single active agent is a treatment for an inflammatory-based disease or disorder.

In another embodiment is a method of administering CaPterin-6-COOH with one or more additional therapies. In another embodiment the co-administration of CaPterin-6-COOH with one or more additional therapies has an increased therapeutic efficacy as compared to administration of the one or more additional therapies without CaPterin-6-COOH. In another embodiment an additional therapy is a nonsteroidal anti-inflammatory drug (NSAID). In another embodiment an additional therapy is acetaminophen. In another embodiment an additional therapy is a disease-modifying anti-rheumatic drug (DMARD). In another embodiment a DMARD is selected from methotrexate, hydroxychloroquine, sulfasalazine, leflunomide, etanercept, certolizumab pegol, adalimumab, infliximab, abatacept, rituximab, and anakinra. In another embodiment an additional therapy is a corticosteroid. In another embodiment an additional therapy is hyaluronic acid therapy. In another embodiment an additional therapy is arthroplasty. In another embodiment an additional therapy is osteotomy.

In another embodiment is a method of administering a composition comprising CaPterin-6-COOH to a human subject. In another embodiment is a method of administering a composition comprising CaPterin-6-COOH to a canine subject.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the X-ray crystallographic structure of dipterinyl calcium pentahydrate (DCP).

FIGS. 2A, 2B, 2C, and 2D depict the oxidative cleavage of the folic acid side chain by chelated calcium ions to yield various pterins.

FIGS. 3A, 3B, and 3C depict the mass spectrometry analysis of 1 mg folic acid +75 mg CaCl₂·2H₂O in 100 ml H₂O.

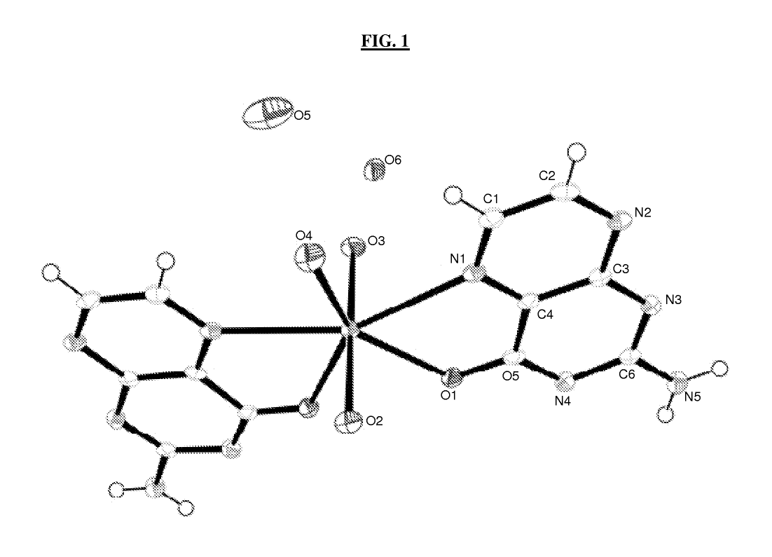

### DETAILED DESCRIPTION OF THE INVENTION

A new chemical moiety identified by mass spectrometry to be CaPterin-6-carboxylic acid has been found to exhibit similar and more potent therapeutic efficacy as compared to CaPterin, DCP and CaFolate in a canine arthritic model. Dosing issues remain to be elucidated.

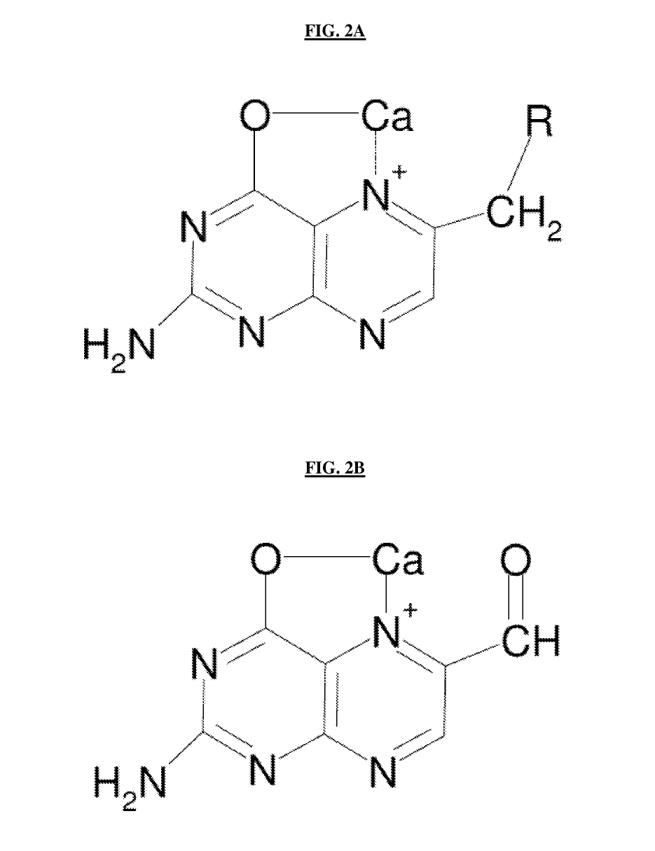

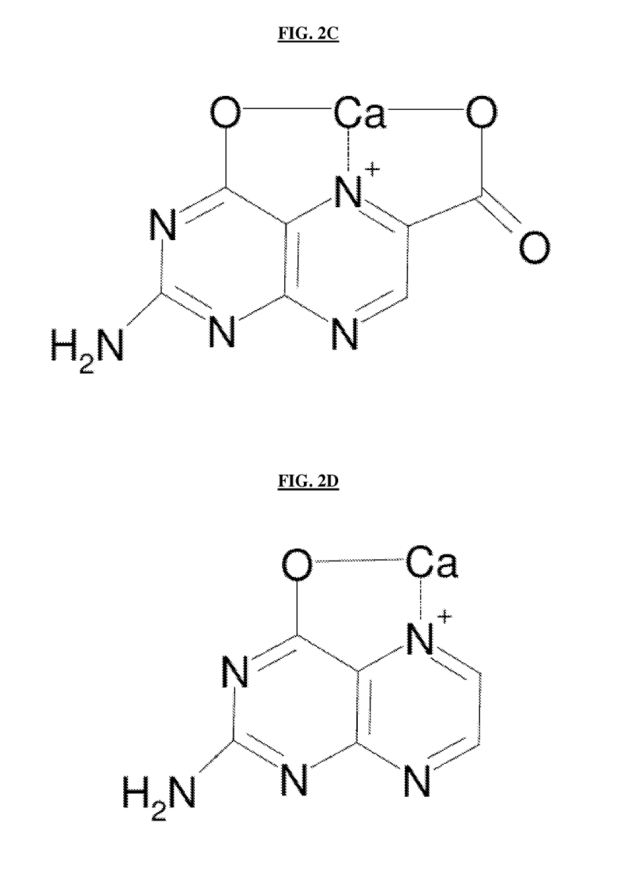

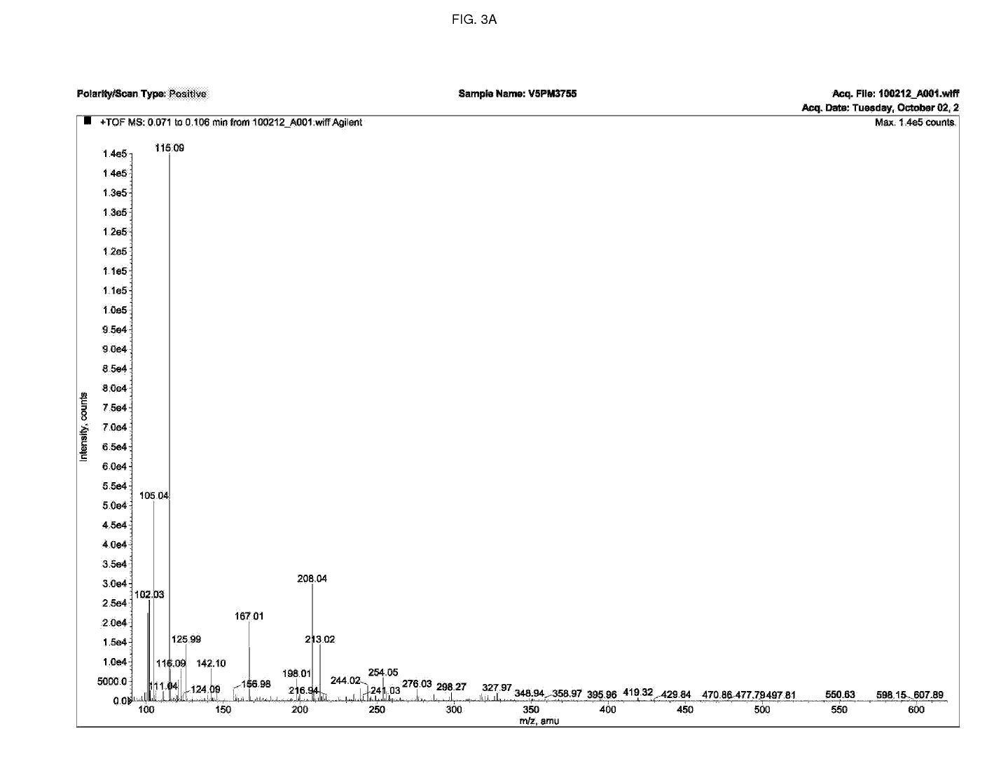

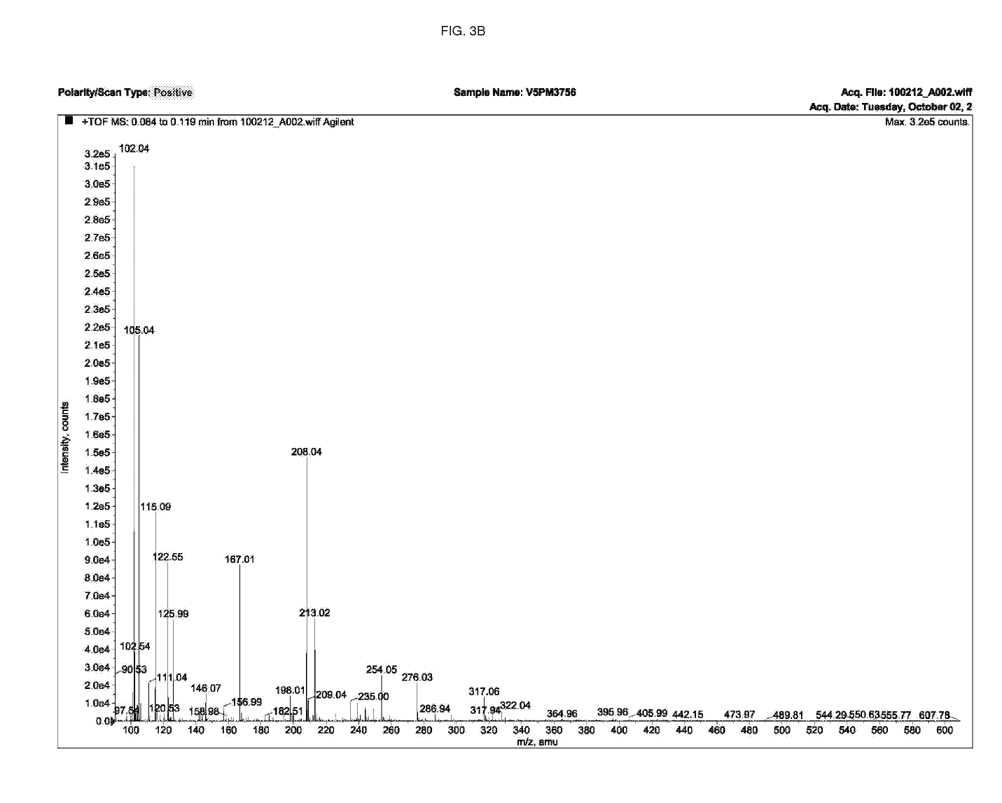

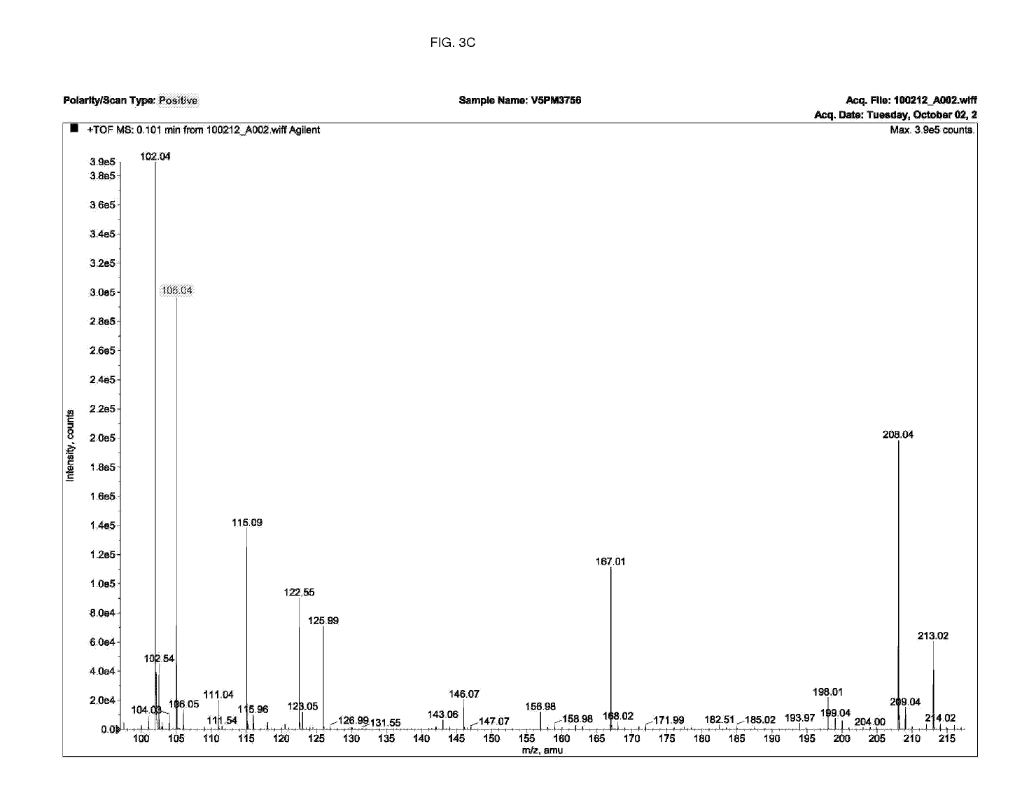

The structure of CaFolate has the same hetero-aromatic ring as CaPterin (FIG. 2). It has been previously observed that under acidic conditions, UV-irradiation of folic acid (pteroylglutamic acid) undergoes successive oxidative cleavage to pterin-6-aldehyde, then to pterin-6-carboxylic acid, and finally to pterin as the end product [4]. FIGS. 3A, 3B and 3C show mass spectra of 1 mg folic acid +75 mg CaCl2•2H2O in 100 ml H2O.

Mass spectrometry analysis performed by the author of 1 mg folic acid +75 mg CaCl₂·2H₂O in 100 ml H₂O showed that Ca⁺² ions (Lewis acids) predominantly drive the formation of pterin-6-carboxylic acid (MW⁺ 208.04) from CaFolate (FIG. 2).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### REFERENCES

1. Moheno, P., et al., Cytokine and IDO metabolite changes effected by calcium pterin during inhibition of MDA-MB-231 xenograph tumors in nude mice. Int J Pharm, 2008. 355(1-2): p. 238-48.

2. Moheno, P., W. Pfleiderer, and D. Fuchs, Plasma cytokine concentration changes induced by the antitumor agents dipterinyl calcium pentahydrate (DCP) and related calcium pterins. Immunobiology, 2009. 214(2): p. 135-41.

3. Moheno, P. B., Calcium pterin as an antitumor agent. Int J Pharm, 2004. 271(1-2): p. 293-300.

4. Lowry, O. H., O. A. Bessey, and E. J. Crawford, Photolytic and enzymatic transformations of pteroylglutamic acid. J Biol Chem, 1949. 180(1): p. 389-98.

5. Ziegler, I., Pterins and the regulation of lymphocyte activation on the mode of xanthopterin action. Hoppe Seylers Z Physiol Chem, 1984. 365(6): p. 667-73.

6. Ziegler, I., Production of pteridines during hematopoiesis and T-lymphocyte proliferation: potential participation in the control of cytokine signal transmission. Med Res Rev, 1990. 10(1): p. 95-114.

7. Ziegler, I., U. Hamm, and J. Berndt, Participation of pterins in the control of lymphocyte stimulation and lymphoblast proliferation. Cancer Res, 1983a. 43(11): p. 5356-9.

8. Ziegler, I., U. Hamm, and J. Berndt, Pterins as functioning constituents of the lymphokine system. In: Curtius, H. Ch., Pfleiderer, W., Wachter, H. (Eds), Biochemical and Clinical Aspects of Pteridines, 1983b. 2(Walter de Gruyter & Co., Berlin, New York): p. 185-193.

9. Ziegler, I. and U. Schwulera, Modulation of interleukin 2 high-affinity binding by lymphocyte-derived tetrahydrobiopterin: pterins as potential participants in the control of interleukin 2 receptor assembly. J Cell Biochem, 1989. 41(2): p. 103-12.

10. Ziegler, I., U. Schwulera, and J. Ellwart, Pteridines are produced during interleukin 2-induced T-cell proliferation and modulate transmission of this signal. Exp Cell Res, 1986. 167(2): p. 531-8.

11. Ziegler, I., et al., Modulation of interleukin 2 activity by lymphocyte-derived tetrahydrobiopterin. Naturwissenschaften, 1985. 72(6): p. 330-1.

12. Stea, B., et al., Urinary excretion levels of unconjugated pterins in cancer patients and normal individuals. Clin Chim Acta, 1981. 113(3): p. 231-42.

13. Moheno, P., J. Morrey, and D. Fuchs, Effect of dipterinyl calcium pentahydrate on hepatitis B virus replication in transgenic mice. J Transl Med, 2010. 8: p. 32.

14. Nikoulina, S. E., D. Fuchs, and P. Moheno, Effect of Orally Administered Dipterinyl Calcium Pentahydrate (DCP) on Oral Glucose Tolerance in DIO Mice. Diabetes, Metabolic Syndrome and Obesity: Targets and Therapy, 2012. 5: p. 43-47.

15. Sakala, I. G., et al., Dipterinyl Calcium Pentahydrate Inhibits Intracellular Mycobacterial Growth in Human Monocytes via the C-C Chemokine MIP-1beta and Nitric Oxide. Infect Immun, 2013. 81(6): p. 1974-83.

1. A method of treating an inflammatory-based disease or disorder in a subject comprising administration of a composition comprising calcium pterin-6-carboxylate (CaPterin-6-COOH).

2. The method of claim 1, wherein CaPterin-6-COOH has a greater therapeutic efficacy as compared to administration of calcium pterin.

3. The method of claim 1, wherein CaPterin-6-COOH has a greater therapeutic efficacy as compared to administration of dipterinyl calcium pentahydrate.

4. The method of claim 1, wherein CaPterin-6-COOH has a greater therapeutic efficacy as compared to administration of calcium folate.

5. The method of claim 1, wherein CaPterin-6-COOH is administered through oral, parenteral, intravenous, subcutaneous, intrathecal, intramuscular, buccal, intranasal, epidural sublingual, pulmonary, local, rectal, or transdermal administration.

6. The method of claim 1, wherein the inflammatory-based disease or disorder is arthritis.

7. The method of claim 6, wherein the arthritis is osteoarthritis, rheumatoid arthritis, gout, psoriatic arthritis, lupus, or septic arthritis.

8. The method of claim 1, wherein CaPterin-6-COOH is produced from folic acid and a calcium source.

9. The method of claim 8, wherein the calcium source is calcium chloride.

10. The method of claim 8, wherein the folic acid and the calcium source are administered to a subject, and wherein CaPterin-6-COOH is produced in the subject after administration of the folic acid and calcium source.

11. The method of claim 1, wherein CaPterin-6-COOH modulates indoleamine 2,3-dioxygenase pathways.

12. The method of claim 1, wherein administration of the composition results in the inhibition of indoleamine 2,3-dioxygenase.

13. The method of claim 1, wherein CaPterin-6-COOH is produced by folic acid exposure to UV light yielding intermediates p-aminobenzoylglutamate and 6-FP, which in turn is oxidized to CaPterin-6-COOH.

14. The method of claim 1, wherein CaPterin-6-COOH is administered as a single active agent.

15. The method of claim 1, wherein CaPterin-6-COOH is administered with one or more additional therapies.

16. The method of claim 15, wherein co-administration of CaPterin-6-COOH with one or more additional therapies has an increased therapeutic efficacy as compared to administration of the one or more additional therapies without CaPterin-6-COOH.

17. The method of claim 15, wherein the therapy is a nonsteroidal anti-inflammatory drug.

18. The method of claim 15, wherein the therapy is acetaminophen.

19. The method of claim 15, wherein the therapy is a disease-modifying anti-rheumatic drug (DMARD).

20. The method of claim 19, wherein the DMARD is selected from methotrexate, hydroxychloroquine, sulfasalazine, leflunomide, etanercept, certolizumab pegol, adalimumab, infliximab, abatacept, rituximab, and anakinra.

21. The method of claim 15, wherein the therapy is a corticosteroid.

22. The method of claim 15, wherein the therapy is hyaluronic acid therapy.

23. The method of claim 15, wherein the therapy is arthroplasty.

24. The method of claim 15, wherein the therapy is osteotomy.

25. The method of claim 1, wherein the subject is a human.

26. The method of claim 1, wherein the subject is a canine.
